(19) United States
(12) Patent Application Publication
(10) Pub. No.: US 2008/0208223 A1
(43) Pub. Date: Aug. 28, 2008

(54) CABLE CLAMPING DEVICE AND METHOD OF ITS USE

(76) Inventor: Paul Edward KRAEMER, Seattle, WA (US)

Correspondence Address: WILEY REIN LLP, 1776 K. STREET N.W., WASHINGTON, DC, 20006, US

(21) Appl. No.: 12/033,350

(22) Filed: Feb. 19, 2008

Related U.S. Application Data
(60) Provisional application No. 60/903,347, filed on Feb. 26, 2007.
(60) Provisional application No. 60/903,411, filed on Feb. 26, 2007.

(51) Int. Cl. A61B 17/08; A43C 1/06

(52) U.S. Cl. 606/151; 24/712

(57) ABSTRACT

A cable clamp includes a housing having two substantially parallel and co-planar channels therethrough. At least one restraining member is disposed within the housing between the two channels, also substantially parallel and co-planar therewith. A surgical cable is passed through the first channel, around a bone or other structure, and through the second channel. The restraining member may then be actuated to impinge against the surgical wire or cable, simultaneously constraining the surgical wire or cable within both channels between the restraining member (an inner wall) and the housing (an outer wall). For example, the restraining member may be an expansion member that, when expanded as by driving a wedge between two restraining arms, reduces the size of the channels such that the surgical wire or cable can no longer pass freely therethrough.

### CROSS-REFERENCE TO RELATED APPLICATIONS

This application claims the benefit of U.S. provisional application Nos. 60/903,347 and 60/903,411, both filed 26 Feb. 2007. The foregoing are hereby incorporated by reference as though fully set forth herein.

### BACKGROUND OF THE INVENTION

a. Field of the Invention

The present invention relates to a system for wrapping a wire or cable around an obstructed structure by accessing the structure through a small opening. In particular, the invention relates to a system for the percutaneous or near percutaneous application of surgical wires or cables in orthopedic fracture surgery and related methods. Even more particularly, the present invention relates to devices and methods for securing surgical wires or cables in such procedures.

b. Background Art

Cerclage cables or wires are used in bone fixation procedures, most commonly in periprosthetic fracture applications where there is an intramedullary stem or implant. Cerclage cables encircle a bone or structure and are cinched and clamped to hold portions of the bone together or retain an intramedullary stem or implant in place. The application of these cables typically requires a large incision to permit access to the fracture site and facilitate passing of the cable or wire around the intended structure. Current devices and methods for the application of cables or wires around bone often result in soft tissue damage, stripping, and devascularization at the site of the fracture. What is needed, therefore, is a system and methods for facilitating the passage of a cable or wire around bone through a small incision that minimizes or reduces these disadvantages.

### BRIEF SUMMARY OF THE INVENTION

The present invention meets these and other objectives by providing devices and methods for wrapping and securing a wire or cable around a structure by accessing the structure through a small opening.

Disclosed herein is a device for restraining at least one elongate member, generally including: a housing including substantially parallel first and second channels therethrough; and at least one restraining member disposed within the housing between the first channel and the second channel, the at least one restraining member being configured to restrain at least one elongate member disposed within at least one of the first channel and the second channel. It is desirable for the first channel, the second channel, and the at least one restraining member to be substantially coplanar and substantially parallel. Optionally, at least one of the first channel and the second channel includes an enlarged region dimensioned to receive a cable head.

Typically, the at least one restraining member is a unitary expansion member adjustable between a collapsed state and an expanded state, wherein, when the expansion member is in an expanded state, a size of at least one of, and preferably both of, the first channel and the second channel is reduced.

In some embodiments of the invention, the first channel is defined in part by a first outer engagement surface and the second channel is defined in part by a second outer engagement surface. Further, the at least one restraining member may include: a first restraining arm including a first inner engagement surface opposite the first outer engagement surface and defining in part the first channel; and a second restraining arm including a second inner engagement surface opposite the second outer engagement surface and defining in part the second channel, wherein a spacing between the first restraining arm and the second restraining arm is adjustable. The first restraining arm may also include a first locking surface opposite the first inner engagement surface and the second restraining arm may also include a second locking surface opposite the second inner engagement surface. Optionally, the first inner engagement surface and the second inner engagement surface may be inclined relative to one another. It is also contemplated that the first restraining arm includes a first end and a second end and the second restraining arm includes a first end and a second end, with the first end of the first restraining arm being connected to the first end of the second restraining arm.

Typically, the invention will include an actuator operable to adjust the spacing between the first restraining arm and the second restraining arm. For example, the at least one restraining member may include a tension screw receptacle between the first restraining arm and the second restraining arm, and the actuator may be a tension screw, such as a conical or cylindrical screw, configured to be received within the tension screw receptacle to increase the spacing between the first restraining arm and the second restraining arm.

Also disclosed herein is a surgical clamping system, the system including: a side-loading cable clamping device, including: a housing having substantially parallel first and second channels therethrough, each of the first and second channels being large enough to pass a surgical cable therethrough, the first channel including a first outer engagement surface and the second channel including a second outer engagement surface; a first restraining arm disposed between the first channel and the second channel and defining in part the first channel, the first restraining arm including a first inner engagement surface opposite the first outer engagement surface; a second restraining arm disposed between the first channel and the second channel and defining in part the second channel, the second restraining arm including a second inner engagement surface opposite the second outer engagement surface; and a restraining actuator configured to be inserted into the housing through an actuator receptacle located between the first restraining arm and the second restraining arm, thereby increasing a distance between the first restraining arm and the second restraining arm and decreasing a size of at least one of the first channel and the second channel. The restraining actuator may be a screw, such as a conical or cylindrical screw. It is contemplated that the first restraining arm and the second restraining arm may be connected to each other opposite the actuator receptacle.

Optionally, the system further includes a cable. The cable may include a head that is too large to pass entirely through at least one of the first channel and the second channel. To recess the cable into the cable clamping device, at least one of the first channel and the second channel may include an enlarged region configured to receive the head.

According to a further aspect of the present teachings, a method of clamping an elongate member generally includes the following steps: providing an elongate member; providing a clamping device having a first channel therethrough, a second channel therethrough, and at least one restraining member disposed between the first channel and the second channel; passing the elongate member through the first channel and the second channel such that a first segment of the elongate member is positioned within the first channel and a second segment of the elongate member is positioned within the second channel; and actuating the at least one restraining member to impinge against the first segment of the elongate member and the second segment of the elongate member, thereby restraining the elongate member from moving relative to the clamping device.

The step of actuating the at least one restraining member may include: causing the first segment of the elongate member to be constrained against a first outer wall of the clamping device; and causing the second segment of the elongate member to be constrained against a second outer wall of the clamping device. For example, the step of causing the first segment of the elongate member to be constrained against a first outer wall of the clamping device may include causing the first segment to be constrained between the first outer wall of the clamping device and the at least one restraining member. Similarly, the step of causing the second segment of the elongate member to be constrained against a second outer wall of the clamping device may include causing the second segment to be constrained between the second outer wall of the clamping device and the at least one restraining member.

Typically, the at least one restraining member includes a first restraining arm and a second restraining arm, and the step of actuating the at least one restraining member includes increasing a spacing between the first restraining arm and the second restraining arm, thereby decreasing a size of at least one of, and preferably simultaneously both of, the first channel and the second channel. The spacing between the first restraining arm and the second restraining arm may be increased by driving a wedge, such as a conical screw, between the first restraining arm and the second restraining arm.

In the preferred embodiments of the invention, the elongate member will be tensioned prior to actuating the at least one restraining member. In addition, it is contemplated that the elongate member may be passed around at least one structure to be bound (e.g., bone or bone and plate) prior to actuating the at least one restraining member, as by passing the elongate member around at least one structure to be bound after passing the elongate member through the first channel and before passing the elongate member through the second channel.

An advantage of the present invention is that it facilitates percutaneous or near percutaneous application of surgical wires or cables in orthopedic fracture surgery.

Another advantage of the present invention is that it minimizes the risk of damaging soft tissue structures, stripping, and devascularization at the site of the fracture.

Still another advantage of the present invention is that it simplifies tensioning and completion of a cerclage procedure.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1A is a first flexible member according to one embodiment of the invention.

FIG. 1B is a second flexible member according to one embodiment of the invention.

FIG. 2 depicts first and second flexible members joined at their distal tips.

FIG. 3A illustrates a first flexible member having a concave distal tip and a second flexible member having a convex distal tip.

FIG. 3B depicts distal tips of first and second flexible members having complementary shapes according to one embodiment of the invention.

FIG. 4 illustrates a cross section of a flexible member having an opening in the outer wall.

FIG. 5A illustrates a cross section of a flexible member having an opening and a groove in the outer wall, the opening being located approximately 90 degrees from the groove.

FIG. 5B illustrates a cross section of a flexible member having an opening and a groove in the outer wall, the opening being located approximately 180 degrees from the groove.

FIG. 6 is a perspective view of a flexible member having an opening in the outer wall.

FIG. 7 is a perspective view of a flexible member having an opening and a groove in the outer wall.

FIG. 8 depicts a stylet according to one embodiment of the invention.

FIG. 9 illustrates the tip of a stylet protruding past the distal tip of a flexible member.

FIG. 10 is a schematic illustration of a cable clamp device according to an embodiment of the present invention.

FIG. 11 is a cross-section of the cable clamp device illustrated in FIG. 10 taken along line 11-11.

FIGS. 12-15 illustrate a sequence of steps that may be carried out in using a cable clamp device according to an embodiment of the present invention.

FIG. 16 illustrates a multi-use tool that may be used in accordance with an aspect of the present teachings.

### DETAILED DESCRIPTION OF THE INVENTION

The present invention provides devices and methods for passing wires or cables around a structure that is concealed or otherwise difficult to access. The present invention will be described in connection with the application of surgical wires or cables around bone in percutaneous or near percutaneous orthopedic fracture surgery. A person of skill in the art will appreciate, however, that the invention may be applied in other contexts where it is desirable to wrap a cable, wire or other elongated line, such as a rope or string, around a concealed or obstructed structure.

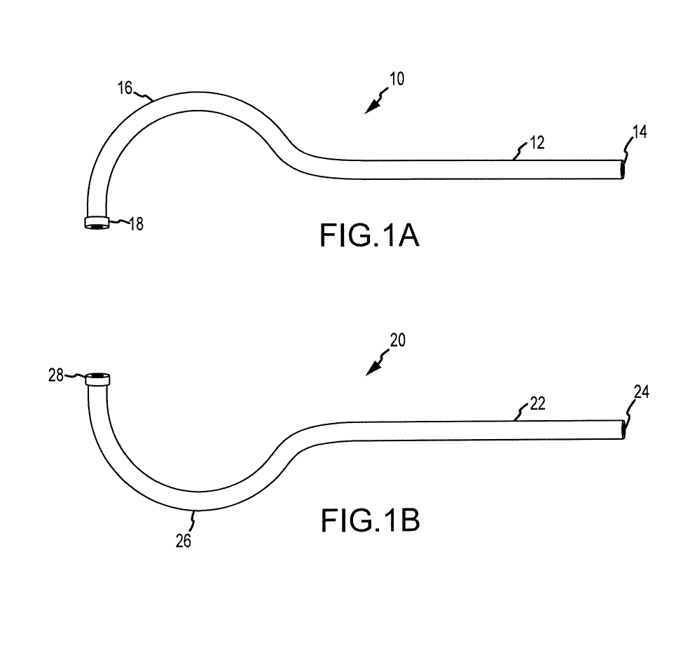

With reference to FIGS. 1A and 1B, a system according to one embodiment of the present invention is shown. FIGS. 1A illustrates a first flexible member 10 and FIG. 1B depicts a second flexible member 20. Each of first and second flexible members 10, 20 includes an outer wall 12, 22 defining a passageway 14, 24 extending through the flexible member and a curved distal portion 16, 26 having a distal tip 18, 28. Flexible members 10, 20 have a generally tubular shape and are preferably made of a semi-rigid material, for example plastics. The diameter of passageways 14, 24 is sufficient for passing a surgical wire or cable therethrough.

Curved distal portions 16, 26 are shaped to curve at least partially around a bone. In preferred embodiments, curved distal portions 16, 26 of first and second flexible members 10, 20 are each shaped to curve approximately 180 degrees around a bone. In alternate embodiments, curved distal portion 16 of first flexible member 10 may be shaped to curve more than 180 degrees around a bone, and curved distal portion 26 of second flexible member 20 may be shaped to curve less than 180 degrees around the bone such that when joined at their distal tips first and second members 10, 20 substantially encircle the bone. For example, curved distal portion 16 of first flexible member 10 may be shaped to curve approximately 230 degrees around a bone and curved distal portion 26 of second flexible member 20 may be shaped to curve approximately 130 degrees around the bone. A person of skill in the art will appreciate that the shape of curved distal portions 16, 26 can be varied in complementary fashion so that the members substantially encircle the bone when joined at their distal tips. The radius of curvature of flexible members 10, 20 may also be varied to accommodate bones of different sizes.

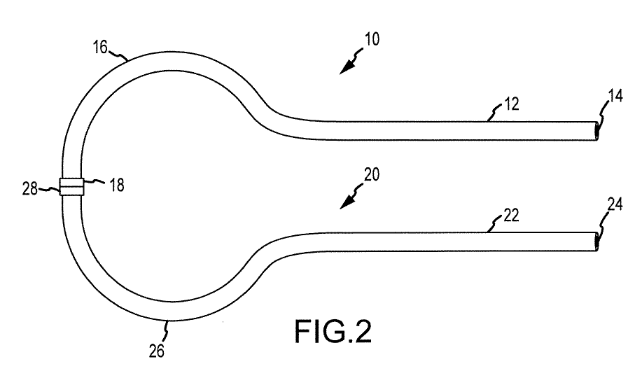

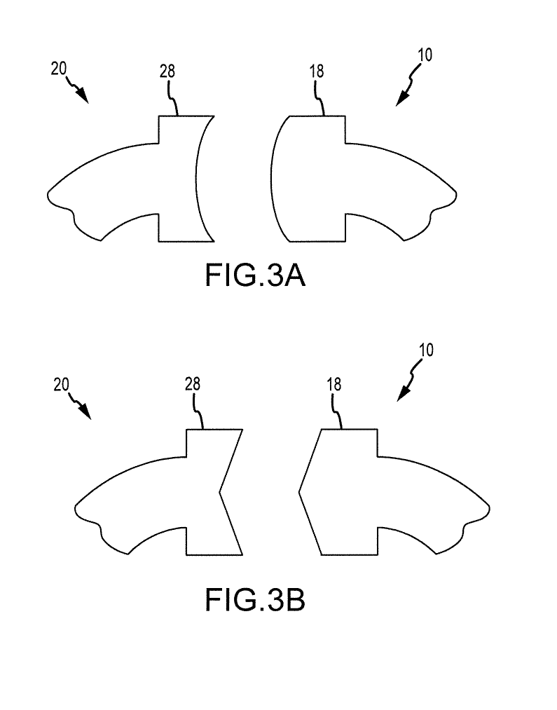

First and second flexible members 10, 20 are preferably used in a complementary fashion whereby each extends at least partially around a bone, as discussed above, and distal tips 18, 28 are joined or connected, as shown in FIG. 2. Preferably, distal tips 18, 28 are shaped complementarily. For example, distal tip 18 may be concave and distal tip 28 may be convex, as shown in FIG. 3A. Alternatively, distal tip 18 may be pointed and distal tip 28 may be indented, as shown in FIG. 3B. A person of skill in the art will appreciate that distal tips 18, 28 can be shaped in any complementary fashion such that distal tips 18, 28 may be joined together on the far side of a bone with minimal manipulation. Distal tips 18, 28 may alternatively include a magnet for connecting distal tips 18, 28 together. In embodiments in which distal tips 18, 28 include magnets, distal tips 18, 28 may be identically shaped, for example, distal tips 18, 28 may be flat. Distal tips 18, 28 are hollow or cannulated to facilitate the passing of a surgical wire or cable from one flexible member to the other. When distal tips 18, 28 are joined, a generally continuous passageway is formed from first flexible member 10 to second flexible member 20.

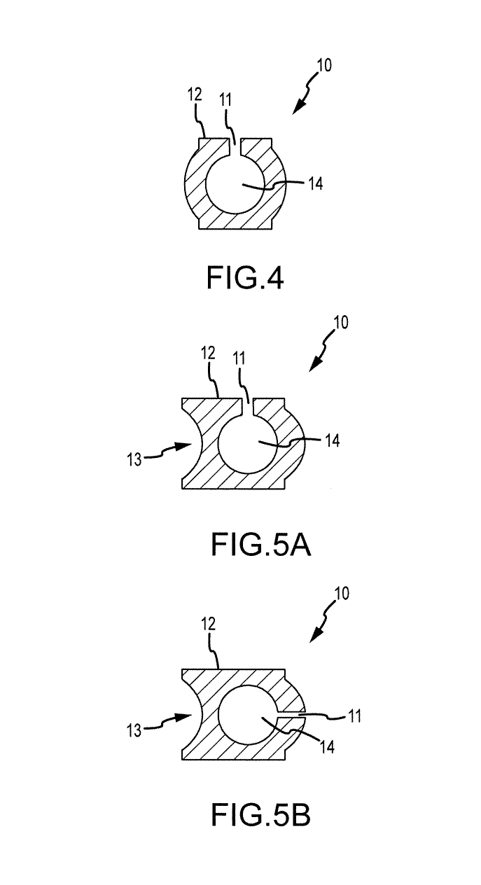

The following features will be discussed with reference to first flexible member 10, but it should be understood that these features may be present in second flexible member 20 without departing from the spirit and scope of the present invention. With reference to FIGS. 4-7, in preferred embodiments, first flexible member 10 includes an opening 11 connected to passageway 14 that extends longitudinally along outer wall 12. Opening 11 preferably extends longitudinally along outer wall 12 of flexible member 10 from a proximal end of the member through the distal tip. Once a surgical wire or cable is in place, flexible member 10 can be removed with minimal force by sliding or pulling it over the wire or cable. Because flexible member 10 is made of a semi-rigid material, opening 11 may stretch or flex to permit flexible member 10 to pass over large articles such as a surgical cable having a diameter larger than the width of opening 11 or a locking or cable crimp mechanism. The width of opening 11 is preferably approximately ½ to ¾ of the thickness of the outer wall at its most narrow surface. The opening 11 should be sufficiently wide to permit a surgical cable or wire to pass through opening 11 when removing flexible member 10, but sufficiently narrow to maintain the cable or wire within passageway 14 of flexible member 10 prior to removing the member.

With reference to FIGS. 5A-B and 7, first flexible member 10 preferably includes a groove 13 extending longitudinally along a portion of outer wall 12. Groove 13 serves as a track for the introduction of second flexible member 20 into the patient along the same soft tissue plane created with first flexible member 10. Groove 13 preferably extends along outer wall 12 to at least the proximal end of curved distal portion 16. Alternatively, groove 13 may extend along the full length of outer wall 12 to distal tip 18. Groove 13 is preferably shaped complementarily to at least a portion of outer wall 22 of second flexible member 20. In preferred embodiments, groove 13 is concave and at least a portion of outer wall 22 of second flexible member 20 is substantially congruent with the concavity of groove 13 such that outer wall 22 of second flexible member 20 fits within groove 13 of first flexible member 10 and is slidable along groove 13. Second flexible member 20 may also include a groove (not shown) in outer wall 22. The groove in outer wall 22 is preferably concave, and at least a portion of outer wall 12 of first flexible member 10 is substantially congruent with the concavity of the groove in second flexible member 20.

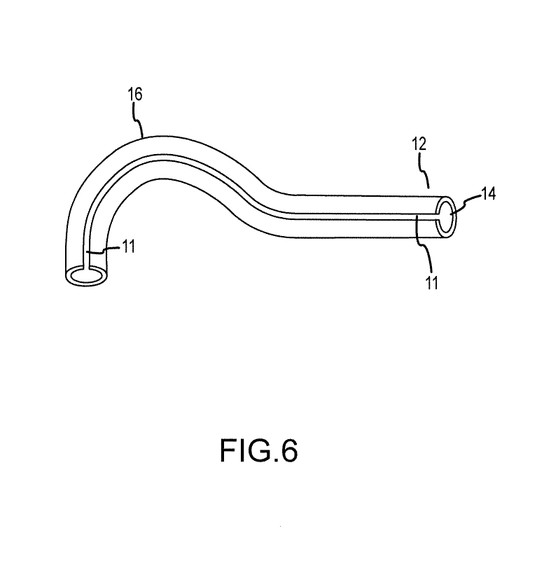

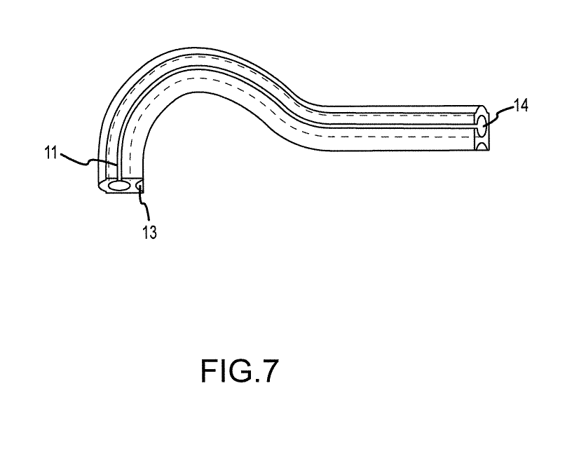

Opening 11 is preferably located on a portion of outer wall 12 of flexible member 10 that does not include groove 13. For example, in FIG. 5A, opening 11 is located approximately 90 degrees from groove 13. In one preferred embodiment, opening 11 is located approximately 90 degrees from groove 13 along the entire length of outer wall 12 and through distal tip 18. Alternatively, as shown in FIG. 5B, opening 11 is located approximately 180 degrees from groove 13 on a convex surface of outer wall 12. In another preferred embodiment, opening 11 begins on the convex surface of outer wall 12, as shown in FIG. 5B, and extends longitudinally along outer wall 12 of flexible member 10 until it reaches the proximal end of curved distal portion 16 (see FIGS. 6 and 7), at which point opening 11 is shifted approximately 90 degrees in either direction. This ensures that a cable or wire inserted through passageway 14 will have a solid surface to track against when passing through curved distal portion 16.

In preferred embodiments, first and second flexible members 10, 20 are identical. In other preferred embodiments, first and second flexible members 10, 20 may be identical except for the shape of distal tips 18, 28, which may be complementary. Preferably, at least one of flexible members 10, 20 includes a groove 13, but it should be understood that both flexible members may include a groove 13.

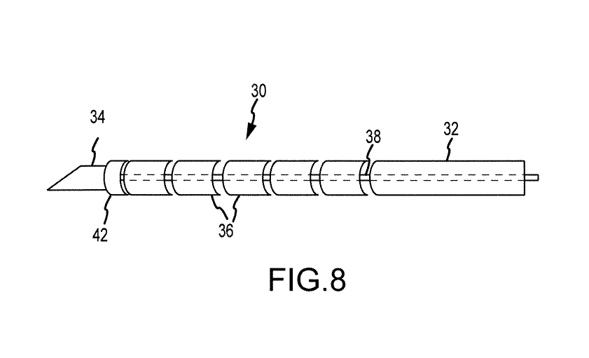

With reference to FIG. 8, a system according to one embodiment of the present invention further includes a stylet 30. Stylet 30 includes a handle 32 at the proximal end, a tip 34 at the distal end and one or more intermediate segments 36 located between handle 32 and tip 34. Stylus 30 is preferably made of a biocompatible metal such as, for example, stainless steel or titanium. In preferred embodiments tip 34 is beveled. More preferably, tip 34 includes a cutting surface, such as a blade or sharp edge. The cutting surface of tip 34 is preferably convex and congruent with the concavity of groove 13 (see FIG. 5). Accordingly, groove 13 may be used as a track to guide tip 34, as will be discussed in more detail herein. Cutting surface of tip 34 allows the stylus to cut a path very near the bone and avoid entrapment of adjacent structures or soft tissue. In addition, tip 34 may be used to facilitate joining or connecting first and second flexible members 10, 20, as described in more detail below.

Stylet 30 further includes a tension cable 38 that interconnects handle 32, intermediate segments 36 and tip 34. Handle 32, intermediate segments 36 and tip 34 are movable relative to one another when tension cable 38 is in a relaxed state, and become substantially rigid or immovable relative to one another when tension cable 38 is tensioned. Stylet 30 is insertable into passageways 14, 24 of flexible members 10, 20. After insertion, cable 38 is tensioned so that stylet 30 may conform to the shape of flexible members 10, 20.

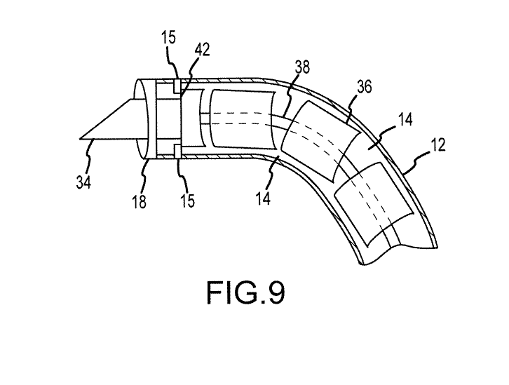

Referring now to FIGS. 8 and 9, in preferred embodiments stylet 30 also includes a lip 42 located between the distal-most intermediate segment 36 and tip 34. Lip 42 may be molded as a single piece with tip 34 or may be a separate piece that is coupled or otherwise attached to tip 34. A person of skill in the art will understand that there are multiple ways to form lip 42. Flexible member 10 may also comprise a ridge 15 near the distal end of passageway 14 (FIG. 9). When inserted into flexible member 10, for example, stylet 30 may be advanced until lip 42 abuts ridge 15. Preferably, ridge 15 is positioned such that tip 34 projects approximately 5-10 mm past distal tip 18 of flexible member 10 when lip 42 and ridge 15 abut. A person of skill in the art will appreciate, however, that the location of ridge 15 may be varied within passageway 14 to permit tip 34 to project less than 5 mm or more than 10 mm past distal tip 18 of flexible member 10. Similarly, the location of lip 42 on tip 34 may be varied to adjust the amount of protrusion of tip 34 from the distal end of flexible member 10. Although ridge 15 is described with reference to flexible member 10, it should be understood that a ridge may also be included in flexible member 20 as described herein without departing from the spirit and scope of the invention.

A method for passing a wire or cable around a structure, such as a bone, is now described. While the method is described in the context of orthopedic fracture surgery, a person of skill in the art will appreciate that the method may be useful in other contexts where it is desirable to wrap a line, such as a wire, rope or string, around a concealed or obstructed structure.

Stylet 30 is inserted into passageway 14 of first flexible member 10 until lip 42 abuts ridge 15. Tension cable 38 is tensioned so that handle 32, tip 34 and intermediate segments 36 become substantially rigid with respect to one another and stylet 30 conforms to the shape of first flexible member 10. First flexible member 10 and stylet 30 are inserted through a single incision to the near surface of a bone. When introducing first flexible member 10 and stylet 30, cutting surface of tip 34 may be used to cut a path very near the bone as described above to avoid entrapment of adjacent structures or soft tissue. First flexible member 10 is manipulated to wrap curved distal portion 16 at least partially around the bone, for example by using the cutting surface of tip 34 to “scrape” around the surface of the bone. Tension cable 38 is then loosened and stylet 30 is removed from passageway 14 of first flexible member 10.

Stylet 30 is inserted into passageway 24 of second flexible member 20. Tension cable 38 is tensioned so that handle 32, tip 34 and intermediate segments 36 become substantially rigid with respect to one another and stylet 30 conforms to the shape of second flexible member 20. Second flexible member 20 is preferably inserted through the same incision as first flexible member 10. Outer wall 22 of second flexible member 20 may be placed in groove 13 of first flexible member 10 and slid along groove 13 to the near surface of the bone. In this manner, second flexible member 20 takes substantially the same path as first flexible member 10 thereby minimizing trauma to the patient. Alternatively, tip 34 of stylet 30 may be placed in groove 13 to guide flexible member 20 to the near surface of the bone along substantially the same path as first flexible member 10. Second flexible member 20 is manipulated to wrap curved distal portion 26 at least partially around the bone from the opposite direction as first flexible member 10. First flexible member 10 and second flexible member 20 are manipulated until distal tips 18, 28 are joined or interlocked on the far side of the bone. Tip 34 of stylet 30 may be advanced through distal tip 28 of second flexible member 20 and into distal tip 18 of first flexible member 10 in a retrograde fashion to facilitate joining distal tips 18, 28 of first and second flexible members together. Tension cable 38 is then loosened and stylet 30 is removed from passageway 24 of second flexible member 20.

A surgical wire or cable is introduced through the proximal end of first flexible member 10, pushed through passageway 14 and into passageway 24 of second flexible member 20, then out the proximal end of second flexible member 20. First and second flexible members 10, 20 are removed by gently pulling them apart. Openings 11 will permit first and second flexible members 10, 20 to slide over the surgical wire or cable and any other instruments that may be present, such as a cable crimp mechanism. The cable or wire may then be tightened in place with instruments designed for percutaneous tightening of wires.

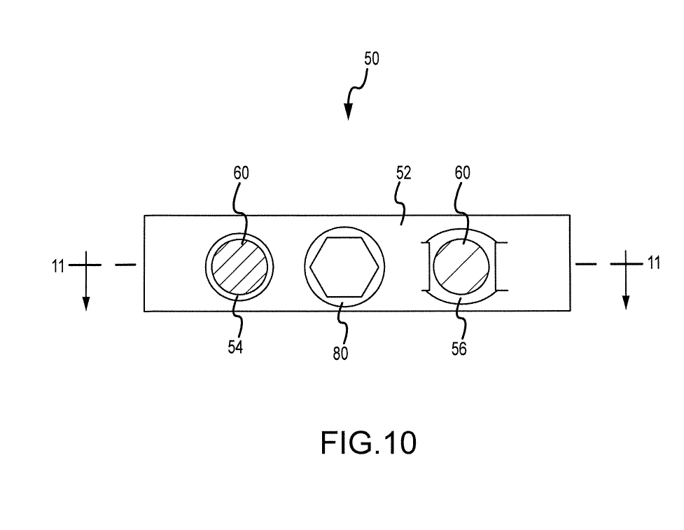

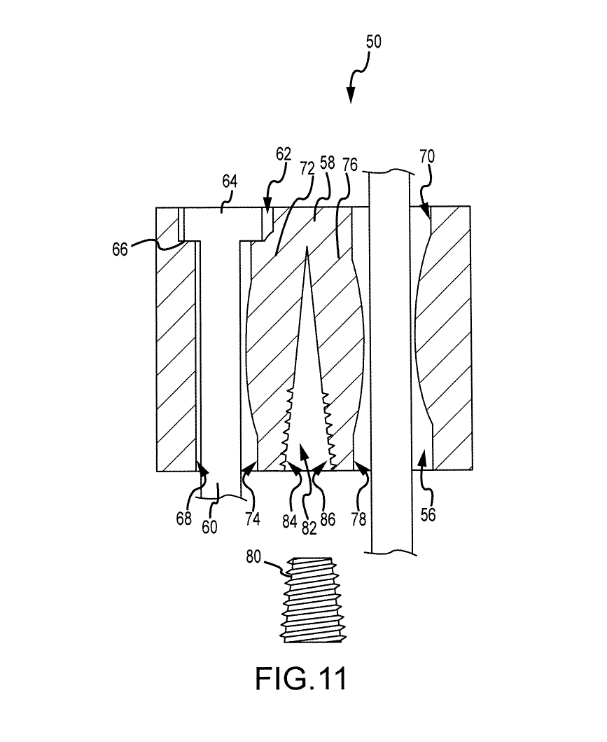

The present teachings also include a cable clamp device such as may be used to restrain the wire, cable, or other elongate member after it has been passed around a structure such as a bone. FIG. 10 schematically illustrates an embodiment of a cable clamp device 50 according to the present teachings. FIG. 11 is a cross-section of cable clamp device 50 taken along line 11-11 in FIG. 10 in order to illustrate various details of cable clamp device 50.

Cable clamp device 50 generally includes a housing 52 having substantially parallel first and second channels 54, 56, respectively, therethrough. At least one restraining member 58 is disposed within the housing between first channel 54 and second channel 56. Restraining member 58 is configured to restrain at least one elongate member, such as a surgical wire or cable 60 as illustrated in FIGS. 10 and 11, disposed within at least one of, and typically both of, first and second channels 54, 56. (For clarity, the intermediate portion of surgical wire or cable 60 is omitted in FIGS. 10 and 11.)

As illustrated in FIGS. 10 and 11, it is desirable for first channel 54, second channel 56, and restraining member 58 to be substantially coplanar and substantially parallel. This co-linear arrangement of channels 54, 56 and restraining member 58 is referred to herein as “side-loading” or “side-operated,” and is advantageous in percutaneous procedures generally, and particularly in percutaneous procedures through a single incision.

Each of first channel 54 and second channel 56 is shaped and dimensioned to pass surgical wire or cable 60 therethrough. It is contemplated that at least one channel (first channel 54 is illustrated) may also include an enlarged region 62 dimensioned to receive a cable head 64. Cable head 64 is larger than first channel 54 such that it cannot pass entirely therethrough. Instead, enlarged region 62 meets first channel 54 at a shoulder 66 upon which cable head 64 rides (that is, rests), thereby preventing surgical wire or cable 60 from pulling entirely through first channel 54 and releasing from cable clamp device 50. Of course, it is within the scope of the present invention for cable head 64 to ride on the outside of housing 52 instead of being recessed into housing 52 by virtue of enlarged region 62.

Preferably, restraining member 58 is a unitary expansion member disposed between first channel 54 and second channel 56. The term “expansion member” is used herein to refer to a member that can be increased in size (e.g., dilated), for example between a collapsed state and an expanded state. Thus, for example, when restraining member 58 is in its expanded state, a size of at least one of, and preferably both of, first and second channels 54, 56 is reduced as further described below.

In some embodiments of the invention, first channel 54 is defined in part by a first outer engagement surface 68, while second channel 56 is defined in part by a second outer engagement surface 70. Restraining member 58 similarly includes a first restraining arm 72 having a first inner engagement surface 74 and a second restraining arm 76 having a second inner engagement surface 78. First inner engagement surface 74 is opposite first outer engagement surface 68 and defines in part first channel 54. Similarly, second inner engagement surface 78 is opposite second outer engagement surface 70 and defines in part second channel 56.

The spacing between first restraining arm 72 and second restraining arm 76 is preferably adjustable via an actuator. In some embodiments of the invention, the actuator may be a tension screw 80 configured to be received within a tension screw receptacle 82 between first and second restraining arms 72, 76. Tension screw 80 may be wedge-shaped (e.g., conical or frusto-conical, referred to herein collectively as “conical”), cylindrical, or any other suitable shape. Preferably, first and second restraining arms 72, 76 are connected to each other opposite tension screw receptacle 82, though it is within the scope of the invention for first and second restraining arms 72, 76 to be disconnected from each other and “free-floating” between first and second channels 54, 56.

First restraining arm 72 includes a first locking surface 84 opposite first inner engagement surface 74 while second restraining arm 76 includes a second locking surface 86 opposite second inner engagement surface 78. Optionally, first and second locking surfaces 84, 86 may be inclined relative to each other such that as tension screw 80 is inserted or threaded into tension screw receptacle 82, first and second restraining arms 72, 76 are wedged apart, simultaneously reducing the size of first channel 54 and second channel 56. Of course, other configurations and arrangements of restraining member 58 and the associated actuator are contemplated.

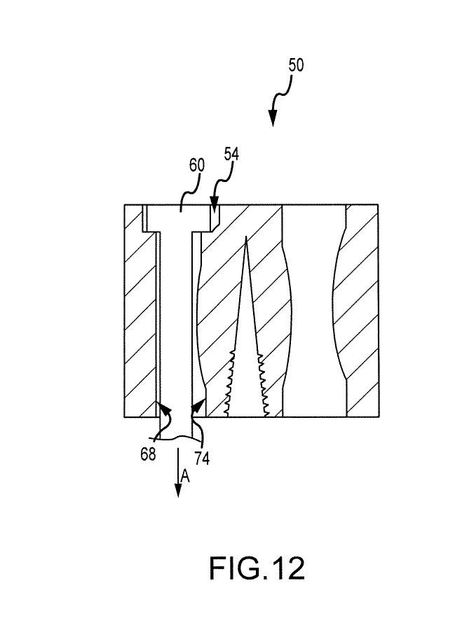

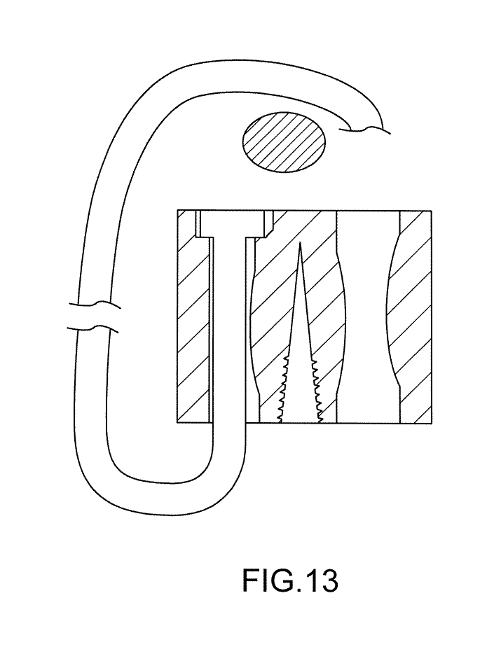

One exemplary use of cable clamp device 50 is illustrated in FIGS. 12-15. An elongate member, such as surgical wire or cable 60, is passed through first channel 54 until cable head 64 is riding on shoulder 66 such that surgical wire or cable 60 can be placed in tension. A first segment of surgical wire or cable 60 will be positioned within first channel 54 between first outer engagement surface 68 and first inner engagement surface 74. This is illustrated in FIG. 12.

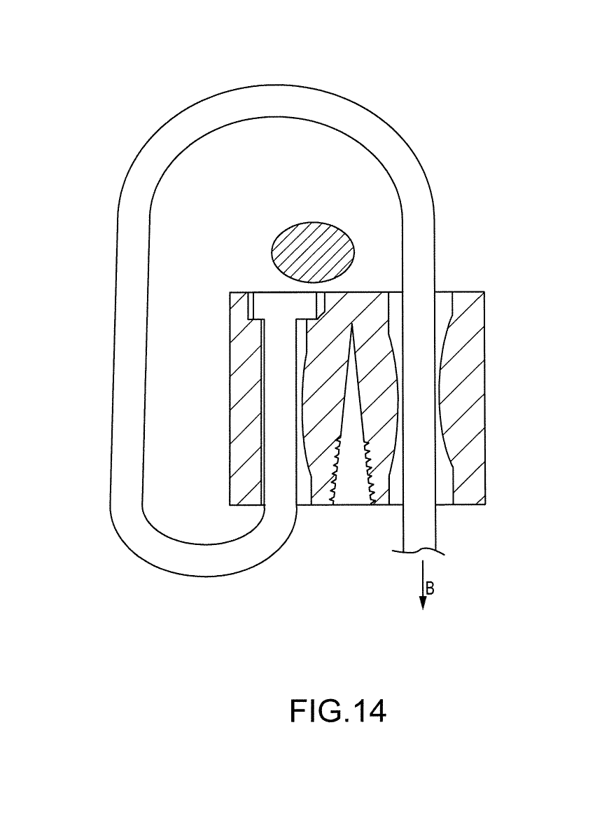

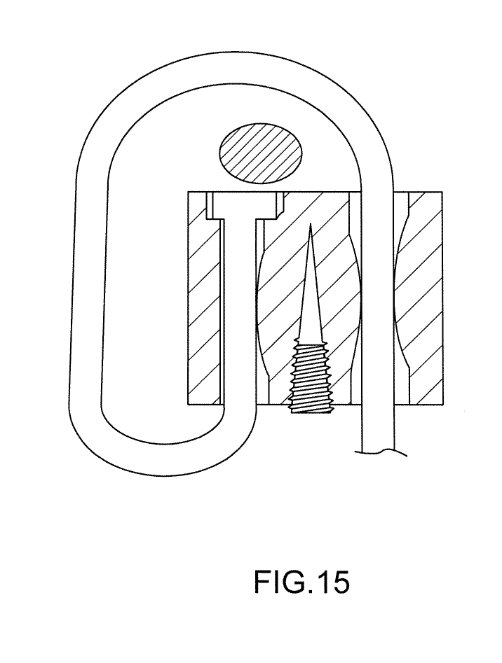

The elongate member is then passed around at least one structure to be bound (e.g., a bone or a bone and a plate), for example using the devices described above, as schematically illustrated in FIG. 13. (For clarity, the intermediate section of surgical wire or cable 60 is illustrated in FIGS. 13-15 as a thin line.) Once the elongate member has traveled the perimeter/circumference of the structure to be bound, it is passed through second channel 56 such that a second segment of the elongate member is positioned between second outer engagement surface 70 and second inner engagement surface 78. This is the configuration illustrated in FIG. 14. The elongate member may then be tensioned as desired by pulling in the direction of arrow “B” (e.g., by using the multi-use tool described below).

As illustrated in FIG. 15, restraining member 58 may then be actuated to impinge against the first segment of the elongate member within first channel 54 and the second segment of the elongate member within second channel 56. For example, a wedge, such as conical tension screw 80, may be driven into (e.g., threaded, as by use of the multi-use tool described herein) tension screw receptacle 82 between first and second restraining arms 72, 76. This restrains the elongate member from moving relative to cable clamp device 50, such as by simultaneously causing the first segment of the elongate member to be constrained between first restraining arm 72 and first outer engagement surface 68 and the second segment of the elongate member to be constrained between second restraining arm 76 and second outer engagement surface 70.

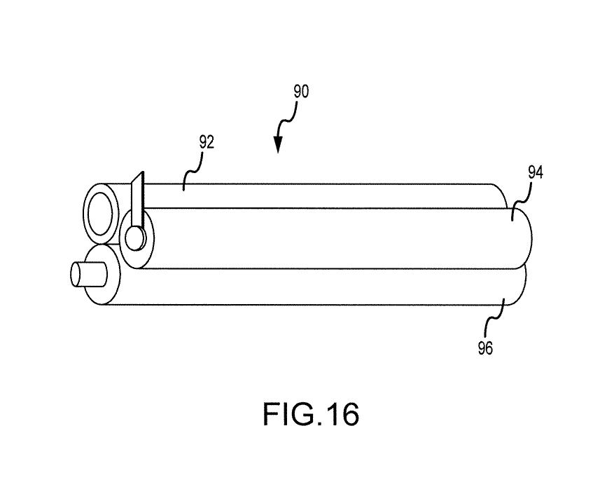

FIG. 16 illustrates a multi-use tool 90 that may be utilized to good advantage in conjunction with the cable passing and clamping devices described herein. Multi-use tool 90 generally includes a tensioning tool 92, a cutting tool 94, and an actuating tool 96. One of skill in the art will appreciate that multi-use tool 90 may include suitable handles (not shown) for utilizing tensioning tool 92, cutting tool 94, and actuating tool 96 as described below.

Tensioning tool 92 receives the free end of the elongate member after it has passed through second channel 56. Analogous to pushing a knot while suturing, tensioning tool 92 pushes cable clamp device 50 towards bone while pulling on the elongate member. In this fashion, tension may be applied to the elongate member until it is snugly around bone with cable clamp device 50 sitting on bone.

Actuating tool 96 is preferably positioned relative to tensioning tool 92 such that, when the elongate member is passed through tensioning tool 92, actuating tool 96 is aligned with tension screw 80. Actuating tool 96 is configured to mate with the actuator (e.g., it may include complementary devices, such as a hex-head screwdriver to mate with a hex-head tension screw 80 as illustrated in FIG. 10). Preferably, actuating tool 96 will be capable of a sufficient degree of translation within multi-use tool 90 without completely separating from multi-use tool 90. Of course, it is within the scope of the invention for actuating tool 96 to be separable from multi-use tool 90.

When the desired tension is achieved in the elongate member through the use of tensioning tool 92, actuating tool 96 may be used to actuate restraining member 58 to constrain the elongate member within first and second channels 54, 56. Cutting tool 94 may then be used to transect the elongate member at the level of cable clamp device 50.

Although several embodiments of this invention have been described above with a certain degree of particularity, those skilled in the art could make numerous alterations to the disclosed embodiments without departing from the spirit or scope of this invention. For example, it is contemplated that friction between the surgical cable or wire and the housing of the cable clamp device may be increased through the use of cleats, serrated nails, and the like.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A device for restraining at least one elongate member, the device comprising:
a housing including substantially parallel first and second channels therethrough; and
at least one restraining member disposed within the housing between the first channel and the second channel, the at least one restraining member being configured to restrain at least one elongate member disposed within at least one of the first channel and the second channel.

2. The device according to claim 1, wherein the first channel, the second channel, and the at least one restraining member are substantially coplanar.

3. The device according to claim 2, wherein the first channel, the second channel, and the at least one restraining member are substantially parallel.

4. The device according to claim 1, wherein at least one of the first channel and the second channel includes an enlarged region dimensioned to receive a cable head.

5. The device according to claim 1, wherein the at least one restraining member comprises a unitary expansion member adjustable between a collapsed state and an expanded state, wherein, when the expansion member is in an expanded state, a size of at least one of the first channel and the second channel is reduced.

6. The device according to claim 5, wherein, when the expansion member is in an expanded state, a size of both the first channel and the second channel is reduced.

7. The device according to claim 1, wherein the first channel is defined in part by a first outer engagement surface and the second channel is defined in part by a second outer engagement surface, and wherein the at least one restraining member further comprises:
a first restraining arm including a first inner engagement surface opposite the first outer engagement surface and defining in part the first channel; and
a second restraining arm including a second inner engagement surface opposite the second outer engagement surface and defining in part the second channel,
wherein a spacing between the first restraining arm and the second restraining is adjustable.

8. The device according to claim 7, wherein the first restraining arm includes a first locking surface opposite the first inner engagement surface and the second restraining arm includes a second locking surface opposite the second inner engagement surface, the first inner engagement surface and the second inner engagement surface being inclined relative to one another.

9. The device according to claim 7, wherein the first restraining arm includes a first end and a second end and the second restraining arm includes a first end and a second end, the first end of the first restraining arm being connected to the first end of the second restraining arm.

10. The device according to claim 7, further comprising an actuator operable to adjust the spacing between the first restraining arm and the second restraining arm.

11. The device according to claim 7, wherein the at least one restraining member further comprises a tension screw receptacle between the first restraining arm and the second restraining arm, wherein the device further comprises a tension screw configured to be received within the tension screw receptacle to increase the spacing between the first restraining arm and the second restraining arm.

12. The device according to claim 11, wherein the tension screw comprises a conical tension screw.

13. A surgical clamping system, the system comprising:
a side-loading cable clamping device, comprising:
a housing having substantially parallel first and second channels therethrough, each of the first and second channels being large enough to pass a surgical cable therethrough, the first channel including a first outer engagement surface and the second channel including a second outer engagement surface;
a first restraining arm disposed between the first channel and the second channel and defining in part the first channel, the first restraining arm including a first inner engagement surface opposite the first outer engagement surface;
a second restraining arm disposed between the first channel and the second channel and defining in part the second channel, the second restraining arm including a second inner engagement surface opposite the second outer engagement surface; and
a restraining actuator configured to be inserted into the housing through an actuator receptacle located between the first restraining arm and the second restraining arm, thereby increasing a distance between the first restraining arm and the second restraining arm and decreasing a size of at least one of the first channel and the second channel.

14. The system according to claim 13, wherein the restraining actuator comprises a screw.

15. The system according to claim 14, wherein the restraining actuator comprises a conical screw.

16. The system according to claim 13, further comprising a cable.

17. The system according to claim 16, wherein the cable includes a head, the head being too large to pass entirely through at least one of the first channel and the second channel.

18. The system according to claim 17, wherein the at least one of the first channel and the second channel includes an enlarged region configured to receive the head.

19. The system according to claim 13, wherein the first restraining arm and the second restraining arm are connected to each other opposite the actuator receptacle.

20. A method of clamping an elongate member, the method comprising:
providing an elongate member;
providing a clamping device having a first channel therethrough, a second channel therethrough, and at least one restraining member disposed between the first channel and the second channel;
passing the elongate member through the first channel and the second channel such that a first segment of the elongate member is positioned within the first channel and a second segment of the elongate member is positioned within the second channel; and
actuating the at least one restraining member to impinge against the first segment of the elongate member and the second segment of the elongate member, thereby restraining the elongate member from moving relative to the clamping device.

21. The method according to claim 20, wherein the step of actuating the at least one restraining member comprises:
causing the first segment of the elongate member to be constrained against a first outer wall of the clamping device; and
causing the second segment of the elongate member to be constrained against a second outer wall of the clamping device.

22. The method according to claim 21:
wherein the step of causing the first segment of the elongate member to be constrained against a first outer wall of the clamping device comprises causing the first segment to be constrained between the first outer wall of the clamping device and the at least one restraining member, and
wherein the step of causing the second segment of the elongate member to be constrained against a second outer wall of the clamping device comprises causing the second segment to be constrained between the second outer wall of the clamping device and the at least one restraining member.

23. The method according to claim 20, wherein the at least one restraining member includes a first restraining arm and a second restraining arm, and wherein the step of actuating the at least one restraining member comprises increasing a spacing between the first restraining arm and the second restraining arm, thereby decreasing a size of at least one of the first channel and the second channel.

24. The method according to claim 23, wherein the step of increasing a spacing between the first restraining arm and the second restraining arm comprises driving a wedge between the first restraining arm and the second restraining arm.

25. The method according to claim 24, wherein the wedge comprises a conical screw.

26. The method according to claim 20, further comprising tensioning the elongate member prior to the step of actuating the at least one restraining member.

27. The method according to claim 20, further comprising the step of passing the elongate member around at least one structure to be bound prior to the step of actuating the at least one restraining member.

28. The method according to claim 27, wherein the step of passing the elongate member around at least one structure to be bound occurs after passing the elongate member through the first channel and before passing the elongate member through the second channel.

29. The method according to claim 27, wherein the at least one structure to be bound comprises a bone.

30. The method according to claim 29, wherein the at least one structure to be bound further comprises a plate.

31. The method according to claim 20, wherein the first channel and the second channel are substantially parallel and coplanar, and wherein the first channel, the second channel, and the at least one restraining member are substantially coplanar.

32. The method according to claim 20, wherein the step of actuating the at least one restraining member to impinge against the first segment of the elongate member and the second segment of the elongate member comprises actuating the at least one restraining member to simultaneously impinge against the first segment of the elongate member and the second segment of the elongate member.
